(19) United States
(12) Patent Application Publication
(10) Pub. No.: US 2001/0038848 A1
(43) Pub. Date: Nov. 8, 2001

(54) Implantable tissues infused with growth factors and other additives

(76) Inventors: Russell S. Donda, Alachua, FL (US); Tom Sander, Alachua, FL (US); Jamie M. Grooms, Alachua, FL (US)

Correspondence Address: Timothy H. Van Dyke, Bencen & Van Dyke, P.A., 1630 Hillcrest Street, Orlando, FL, 32803, US

(21) Appl. No.: 09/789,292

(22) Filed: Feb. 20, 2001

Related U.S. Application Data
(60) Provisional application No. 60/183,468, filed on Feb. 18, 2000.
(60) Provisional application No. 60/197,477, filed on Apr. 17, 2000.
(60) Provisional application No. 60/184,203, filed on Feb. 22, 2000.

(51) Int. Cl. A61K 45/00

(52) U.S. Cl. 424/423; 424/93.7

(57) ABSTRACT

Disclosed herein are novel configurations of tissue designed for simple implementation and use in various medical applications. Specifically exemplified herein are sections of tissue shaped and provided in tape form or patch form, and kits and methods implementing the same. Alternatively, there is disclosed sections of tissue having osteogenic properties and especially adapted for use in the repair of bone defects, diseases or injuries.

### CROSS-REFERENCE TO RELATED APPLICATIONS

The subject application claims the benefit under 35 USC §119 of Provisional Application Nos. 60/183,468 filed Feb. 18, 2000; 60/197,477 filed Apr. 17, 2000; and 60/184,203 filed Feb. 22, 2000.

### BACKGROUND OF THE INVENTION

A need exists in the medical arts for a readily usable, non-immunogenic biomaterial for various medical applications, such as bone fracture fixation, guided tissue regeneration, and repair of injuries to soft or hard tissues and organs caused by trauma, disease, or otherwise.

### SUMMARY OF THE INVENTION

The subject invention pertains to novel configurations of tissue which lend themselves to ready and simple use for various medical applications. According to a specific aspect, the subject invention relates to tissue shaped into the form of tape. Preferably, the tissue tape is provided as a spool, whereby sections or an amount of tissue tape can be easily peeled off as needed. Alternatively, the subject invention pertains to tissue configured as several separate sections or “patches”, and preferably provided together in a container, whereby sections can easily be removed from the container and ready to use. Preferably still, the tissue patches or tissue tape have a bioadhesive disposed on at least one side. The subject tissue tape and patches may also have a medically useful additive infused therein.

These and other advantageous aspects of the subject invention are described below.

### DESCRIPTION OF THE PREFERRED EMBODIMENTS

The subject invention is directed to configurations of tissue obtained from a human or nonhuman animal. The term “tissue” as used herein refers to any animal tissue types including, but not limited to, bone, bone marrow, fibrous connective tissue, yellow elastic connective tissue, cartilage, muscle, vasculature, epidermis, and dermis. Tissue for use in accord with the principles of the subject invention can be human and/or nonhuman tissue. Preferably, the tissue used for producing the subject tape is skin or fibrous or elastic connective tissue (e.g., fascia lata, tendons, peritoneum, dura mater, cartilage, pericardium or ligaments).

According to a specific aspect, the subject invention pertains to tissue that is processed and shaped, cut, and/or stretched into an elongated tape form and “rolled” into a spool. Processing can include sterilizing and/or decellularization of crude tissue. Cleaning and sterilization of tissue can be accomplished by techniques known in the art, such as accordingly to procedures taught in U.S. Pat. Nos. 5,993,844; 5,820,581; 5,797,871; 5,556,379; 5,513,662; 5,333,626; and 5,095,925. See also U.S. patent application Ser. Nos. 09/191,232; 09/390,174; and 09/378,527. The subject tape can be peeled from the spool in the desired size and used in various medical applications, including, but not limited to, bone fracture fixation, guided tissue regeneration, and repair trauma injuries to soft tissue and organs.

According to another aspect, the subject tape can be infused with medically/surgically useful substances. Those skilled in the art will readily appreciate appropriate substances to infuse into the subject tape based on the intended medical application. The terms “infuse” or “infused” are used herein in their broad sense and are intended to mean any association with the tape whereby a substance is allowed to effectuate its intended beneficial effect, whether it be released or whether contact with the tape is maintained. Examples of substances useful in accord with the subject invention include, e.g., collagen and insoluble collagen derivatives; gelatin; hydroxyapatite, etc., and soluble solids and/or liquids dissolved therein, e.g., antiviricides, particularly those effective against viruses such as HIV and hepatitis; antimicrobials and/or antibiotics such as erythromycin, bacitracin, neomycin, penicillin, polymyxin B, tetracyclines, viomycin, chloromycetin and streptomycins, cefazolin, ampicillin, azactam, tobramycin, clindamycin and gentamycin, etc.; amino acids, magainins, peptides, vitamins, inorganic elements, co-factors for protein synthesis; hormones; endocrine tissue or tissue fragments; enzymes such as collagenase, peptidases, oxidases, etc.; polymer cell scaffolds with parenchymal or other cells; surface cell antigen eliminators; angiogenic or angiostatic drugs and polymeric carriers containing such drugs; collagen lattices; biocompatible surface active agents; antigenic agents; cytoskeletal agents; cartilage fragments, living cells such as chondrocytes, bone marrow cells, mesenchymal stem cells, natural extracts, tissue transplants, bioadhesives, growth factors, growth hormones such as somatotropin; bone digestors; antitumor agents; fibronectin; cellular attractants and attachment agents; immuno-suppressants; permeation enhancers, e.g., fatty acid esters such as laureate, myristate and stearate monoesters of polyethylene glycol, enamine derivatives, alpha-keto aldehydes, etc.; nucleic acids; and, bioerodable polymers such as those disclosed in U.S. Pat. Nos. 4,764,364 and 4,765,973. The amounts of such optionally added substances can vary widely with optimum levels being readily determined in a specific case by routine experimentation.

According to a preferred aspect, the tape of the subject invention is infused with one or more growth factors. The term “growth factor” as used herein refers to a polynucleotide molecule, polypeptide molecule, or other related chemical agent that is capable of effectuating differentiation of cells. Examples of growth factors as contemplated for use in accord with the teachings herein include an epidermal growth factor (EGF), transforming growth factor-alpha (TGF.alpha.), transforming growth factor-beta (TGF-.beta.), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), cartilage derived morphogenetic protein (CDMP), and/or platelet derived growth factor (PDGF). Growth factors for use in accord with the teachings herein can be extracted from allograft, xenograft and/or autograft tissue, or can be produced by recombinant/genetic means, or be encoded by nucleic acids associated with appropriate transcriptional and translational elements associated with the tape.

An alternative embodiment of the subject invention is directed to a section of tissue, and method of using the same, wherein the section of tissue has infused therein growth factors having properties related to the regeneration of tissue. Preferably, the section of tissue comprises dermal tissue, and more preferably is provided in patch form. According to a particular aspect, the subject invention is directed to a method comprising obtaining a section of tissue and infusing it with growth factors that comprise the ability to stimulate generation of specific tissues; and implanting said section in an area of a patient in need of repair of such specific tissues, or further generation of such tissues. Preferably, the method comprises obtaining a section of tissue and infusing it with growth factors that comprise the ability to stimulate the generation of vascular tissue, e.g., VEGF, ECGF, TGF, FGF, or combinations thereof, as well as later developed growth factors having similar activity. The section of tissue infused with such growth factors is then implanted into a patient in an area where vascular tissue is damaged and in need of repair, or where the generation of further vascular tissue would be beneficial. In a preferred embodiment, a section of the subject tape infused with vascular tissue generating growth factors is surgically joined to a damaged vein or artery, such as by suturing of the section to the vein or artery or other conventional methods used in the surgical arts. Even more preferred, the damaged artery or vein is located on the heart. An alternative aspect of this embodiment pertains to a method comprising obtaining a section of tissue and infusing it with growth factors having the ability to stimulate generation of muscle or connective tissue; and implanting the section in a patient in need thereof.

According to a further aspect, the subject tissue tape can have a bioadhesive disposed on at least one side to aid in the attachment to the desired area. In the present context the term “a bioadhesive substance” is broadly defined as a material that is capable of being bound to a biological membrane, and retained on that membrane for an extended period of time. Accordingly, “bioadhesion” is the attachment of a material to a biological substrate such as a biological membrane. Examples of bioadhesives that can be used in accord with the teachings herein, include but are not limited to, fibrinogen, fatty acid ester adhesives as disclosed in U.S. Pat. No. 5,955,502, gelatin/aldehyde adehesives as disclosed in U.S. Pat. No. 6,007,613, starch adhesives as disclosed in U.S. Pat. No. 5,804,209. See U.S. Pat. Nos. 4,253,460; 4,740,365; and 4,772,470 for examples of other bioadhesives suitable for use with the subject invention.

The subject tissue tape can also comprise a backing layer, which will facilitate peeling the tissue tape from a spool. This backing layer is particularly helpful when the tissue tape comprises a bioadhesive disposed on its surface. The backing layer can be comprised of a non-stick substance such as TEFLON(registered trademark), or other commercially available plastic and/or polymeric materials. Suitable materials will readily be appreciated by those skilled in the art. The backing material may also be comprised of a natural substance, for example, processed tissue.

In an alternative embodiment, graft tissues are treated with Platelet Rich Plasma (PRP), or growth factors isolated from PRP. PRP obtained from autograft blood has been shown to increase the rate of healing of autogenic grafts. Current methods of applying PRP to such grafts involves the removal of blood from a patient (plasmapheresis), centrifuging the blood, drawing off the PRP layer, and applying the PRP to the graft, which occurs just prior to surgery. There is a need in the art to alleviate the costs and inefficiencies involved with the current methods. Accordingly, in a further embodiment of the subject invention, provided is a method of obtaining an allograft and/or xenograft source of PRP for use in graft implantation. In a specific embodiment, the PRP is obtained by procuring blood from a cadaveric donor (such as by conventional exsanguination techniques) or procuring blood (preferably expired blood as to avoid depletion of blood earmarked for other purposes) from blood banks, and centrifuging the obtained blood to separate the PRP from other blood components via conventional methods. Preferably, PRP is obtained from a cadaveric donor. The isolation of PRP from sources other than autogenous (recipient) allows for the manipulation and use of the PRP well prior to surgery, whereby the inefficient removal and treatment of blood from the recipient is alleviated.

Furthermore, it is generally believed in the art that the beneficial effects of PRP are due to the presence of various growth factors, such as platelet derived growth factor (PDGF), platelet derived angiogenic growth factor (PDAF), platelet derived epidermal growth factor (PDEGF), and transforming growth factor (TGF-beta). Allogenic and/or xenogenic blood provides a vast and untapped source for PRP and growth factors. In a specific embodiment, platelets are isolated from allogenic and/or xenogenic sources as described above, and growth factors are partially purified or purified from these isolated platelets via conventional methods (see, e.g., U.S. Pat Nos. 4,479,896; 4,861,757; or 4,975,526). As used herein, the term “partially purified” refers to a state of purification above that which is found in nature, or said differently, that is not achievable unless through manipulation by the hand of man. The term “purified” as used herein refers to a state of purification such that in a given sample comprising a given growth factor, the growth factor is 95% or greater, by weight, of the sample. Partially purified growth factors may also be obtained from PRP by the following method:

1. Obtain outdated apheretically purified platelets (platelets present in 60-70 ml plasma). Keep platelets at 4° C.

2. Combine donor platelets into 500 ml centrifuge tubes. Centrifuge 8000×g 20 minutes at 4° C. Remove plasma

3. Add 20 volumes of ice cold sterile saline to platelets and gently resuspend pellet. This step is to remove as much plasma/serum components as possible.

4. Re-centrifuge 8000×g 20 min at 4° C. to repellet platelets.

5. To platelet pellet, add 10 volumes extraction buffer and agitate overnight at 4° C. (12-16 hours).

6. Pellet lysed platelet material by centrifugation at 12,000 rpm 20 minutes 4° C. Remove platelet extract.

Acid Ethanol extraction buffer

45% Ethanol containing 150 ul concentrated HCl for every 50 ml of solution

High salt extraction buffer

100 mM NaH₂PO₄

1.5M NaCl

pH 7.4

Once they are partially purified or purified, the growth factors can be stored and/or distributed in a lyophilized or frozen form. Accordingly, the subject methods allow for the mass production of implants (autogenic, allogenic, and/or xenogenic) that have been treated with PRP, and/or growth factors isolated therefrom, that are readily usable in implantation surgeries, which also decreases the costs and inconvenience associated with conventional methods.

In a preferred embodiment, growth factors obtained from blood, or any other growth factors obtained from other tissues as previously described above, are placed in an easy to use container such as a bottle, vial, bag, etc. made from glass or plastics, or other suitable materials. Providing the subject growth factors as a composition in containers will facilitate the use of the growth factors, for example, for the infusion or other treatment of implants to be implanted into a patient, or for the direct administration of the growth factors into a patient. The choice of carrier material for the growth factor composition is based on biocompatibility, biodegradability, and interface properties. The growth factor composition can be infused into the implant in any suitable manner. For example, the growth factor composition may be injected into the implant. In other embodiments the composition is dripped onto the implant or the implant is soaked in a solution containing an effective amount of the composition to carry out its intended effect. In either case the implant is exposed to the growth factor composition for a period of time sufficient to allow the liquid to thoroughly soak the implant. The growth factors may be provided in freeze-dried form and reconstituted in a pharmaceutically acceptable liquid or gel carrier such as sterile water, physiological saline or any other suitable carrier. The carrier may be any suitable medium capable of delivering the proteins to the implant. Preferably the medium is supplemented with a buffer solution as is known in the art. In one specific embodiment of the invention, growth factors are suspended or admixed in a carrier, such as water, saline, liquid collagen or injectable bicalcium phosphate. The growth factor solution can be dripped into the implant or the implant can be immersed in a suitable quantity of the liquid. In a most preferred embodiment, the growth factor composition is applied to the implant and then lypholized or freeze-dried. The implant/growth factor composition can then be frozen for storage and transport.

Tape Comprising Osteogenic Characteristics

The subject invention is directed to an osteogenic tape comprised of an osteogenic material. The term “osteogenic material” is used herein in its broad sense and refers to a material comprising an osteoinductive substance, osteoconductive substance, chondrogenic substance, or a combination of one or more of the foregoing substances. Examples of osteoconductive materials suitable for use with the subject invention include, but are not limited to, hydroxapatite (HA), tricalcium phosphate (TCP), corticocancellous chips (CCC), bioactive glass, bioactive ceramics, and/or mixtures thereof. Examples of osteoinductive materials suitable for use with the subject invention include, but are not limited to, allograft or xenograft pastes (osteogenic or chondrogenic pastes), demineralized bone matrix (DBM), bone morphogenic protein (BMP), TGF-beta, PDGF, FGF, CDMP, and/or mixtures thereof. In a preferred embodiment, the osteogenic material is combined with a carrier. Accordingly, examples of other osteogenic materials for use with the teachings herein include, but are not limited to, carrier associated Growth Factors, carrier associated mineralized particles, morsellized skin or other tissue, Fibrin powder, Fibrin/plasminogen glue, biomedical plastics, Demineralized Bone Matrix (DBM)/glycerol, cortico cancellous chips (CCC), DBM/pleuronic F127, and DBM/CCC/F127, human tissue/polyesters or polyhydroxy compounds, or polyvinyl compounds or polyamino compounds or polycarbonate compounds or any other suitable viscous carrier. Suitable carriers include, but are not limited to, amylopectin, collagen, gelatin, dextran, agrarose, or combinations thereof.

In a further embodiment, the subject osteogenic tape has disposed on at least one side an inert material, such as a resorbable polymer. The term “inert” as used herein refers to the quality of being non-immunogenic or otherwise does not invoke an undesirable effect once in contact with a tissue. Inert materials contemplated for use herein do not necessarily have osteoinductive or osteoconductive qualities. Examples of inert materials suitable for use with the teachings herein include, but are not limited to, polylactide, poly (alpha-hydroxycarboxylic acids (e.g. poly-D-(−)-3hydroxybutyric acid, poly(lactones), poly(acetals), poly(orthoesters) or poly(orthocarbonates). Further examples include amylopectin, gelatin, collagen, agarose, dextran, or combinations thereof. Alternatively, the osteogenic tape of the subject invention comprises two or more layers, wherein a first layer is covered with a second layer. The second layer (or “backing layer”) can comprise a film like material derived from tissue. The term “tissue” as used herein refers to any animal tissue (human or nonhuman; allograft, xenograft and/or autograft) types including, but not limited to, bone, bone marrow, fibrous connective tissue, yellow elastic connective tissue, cartilage, muscle, vasculature, and skin. Preferably, the tissue used for producing the second layer of the subject tape is skin or fibrous or yellow elastic connective tissue (e.g., fascia lata, tendons, peritoneum, dura mater, pericardium or ligaments).

According to a specific aspect, the tape of the subject iinvention is shaped, cut, and/or stretched into an elongated tape form and “rolled” into a spool. The subject tape can be peeled from the spool in the desired size and used in various medical applications, including, but not limited to, bone fracture fixation, filling of voids in bone, or filling of voids between prostheses and bone, enclosing and protecting an implant site, and repairing trauma injuries to soft tissue and organs. The tape of the subject invention would also be useful in sealing a graft implant site.

Yet another aspect of the subject invention pertains to an osteogenic tape having woven (or otherwise attached) interiorly or exteriorly a support structure such as a mesh, suture, and/or wire material, to help strengthen the tape or otherwise make the tape more suitable for unrolling and “taping.” Examples of materials for use as the support structure include, but are not limited to, inert metals such as titanium; inert and/or bioresorbable polymers; bone, dimeralized bone, and/or human or nonhuman tissue.

According to a further aspect, the subject osteogenic tape can have a bioadhesive disposed on at least one side to aid in the attachment to the desired area. In the present context the term “a bioadhesive substance” is broadly defined as a material that is capable of being bound to a biological membrane or tissue surface, and retained on that membrane or tissue surface for an extended period of time. Accordingly, “bioadhesion” is the attachment of a material to a biological substrate such as a biological membrane. Examples of bioadhesives that can be used in accord with the teachings herein, include but are not limited to, fibrinogen, fatty acid ester adhesives as disclosed in U.S. Pat. No. 5,955,502, gelatin/aldehyde adehesives as disclosed in U.S. Pat. No. 6,007,613, starch adhesives as disclosed in U.S. Pat. No. 5,804,209. See U.S. Pat. Nos. 4,253,460; 4,740,365; and 4,772,470 for examples of other bioadhesives suitable for use with the subject invention.

Those skilled in the art will appreciate, in view of the teachings herein, that the subject osteogenic tape is produced by a number of conventional techniques currently used in the art. For example, the subject osteogenic tape can be prepared by casting the osteogenic material as a dispersion in a solvent onto the backing layer (as described above) and drying the composition to remove the solvent. Alternatively the tape of the subject invention can be formed by pressing the osteogenic material, either in a mold, by extrusion, calendering, or combinations of pressing, extruding and/or calendering, to thereby form the appropriate shape either with or without a backing layer. See U.S. Pat. Nos. 5,997,675; 5,817,395; and 5,810,756 for a discussion of conventional methods for producing films or tapes.

The disclosure of all references cited herein are incorporated by reference to the extent they are not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A biomedical implant derived from tissue, wherein said biomedical implant is shaped in the form of tape.

2. The biomedical implant of claim 1, wherein said tissue is human tissue.

3. The biomedical implant of claim 1, wherein said tissue is bone, fibrous connective tissue, elastic connective tissue, cartilage, muscle, vasculature, skin, mucus membrane, submucosa, pericaridium, or combinations or derivatives thereof.

4. The biomedical implant of claim 3, wherein said tissue is fibrous connective tissue.

5. The biomedical implant of claim 1, wherein said biomedical implant is rolled into a spool.

6. The biomedical implant of claim 1, wherein said biomedical implant is in patch form.

7. The biomedical implant of claim 1, wherein said biomedical implant is infused with an additive.

8. The biomedical implant of claim 6 wherein said additive is collagen and insoluble collagen derivatives; gelatin; hydroxyapatite with or without soluble solids and/or liquids dissolved therein; antiviricides,; antimicrobials; antibiotics; amino acids: magainins: peptides; vitamins; inorganic elements; co-factors for protein synthesis; hormones; endocrine tissue or tissue fragments; synthesizers; enzymes; polymer cell scaffolds with parenchymal cells; surface cell antigen eliminators; angiogenic drugs and polymeric carriers containing such drugs; collagen lattices; biocompatible surface active agents; antigenic agents; cytoskeletal agents; cartilage fragments; living cells; natural extracts; tissue transplants; bioadhesives; growth factors; growth hormones; bone digesters; antitumor agents; fibronectin; cellular attractants and attachment agents; immuno-suppressants; permeation enhancers; nucleic acids; and, bioerodable polymers.

9. The biomedical implant of claim 7, wherein said additive is epidermal growth factor (EGF), transforming growth factor-alpha (TGF.alpha.), transforming growth factor-beta (TGF-.beta.), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), platelet derived growth factor (PDGF), cartilage derived morphogenetic protein (CDMP), or combinations thereof.

10. The biomedical implant of claim 1, wherein an adhesive is disposed on at least one side of said biomedical implant.

11. The biomedical implant of claim 9, wherein said adhesive is fibrinogen, fatty acid ester adhesive, gelatin/aldehyde adhesive, or a combination thereof.

12. The biomedical implant of claim 10, wherein said adhesive is fibrinogen.

13. A method of repairing soft or hard tissues and organs comprising the steps of obtaining a biomedical implant derived from tissue, wherein said biomedical implant is in the form of tape that is rolled into a spool; and peeling said biomedical implant from said spool.

14. The method of claim 12, wherein repairing soft or hard tissues and/or organs comprises tissue and/or fracture fixation, guided tissue regeneration, implanting a spinal tension band, anterior ligament replacement, or providing support to ligaments.

15. A biomedical implant comprising a section of tissue infused with one or more growth factors and/or nucleic acids.

16. The biomedical implant of claim 14 wherein said tissue comprises dermis tissue.

17. The biomedical implant of claim 14 wherein said growth factors have vascular tissue generating properties.

18. The biomedical implant of claim 16 wherein said growth factors are VEGF, TGF, ECGF, FGF, or combinations thereof.

19. A method of repairing damaged tissue, or stimulating the generation of tissue, comprising the steps of obtaining a section of tissue infused with one or more growth factors, and implanting said section into a patient in need thereof.

20. The method of claim 18 wherein said tissue is vascular tissue, and wherein said implanting comprises joining said section to an artery or vein.

21. The method of claim 19 wherein said one or more growth factors have vascular tissue generating properties.

22. The method of claim 20 wherein said one or more growth factors are VEGF, TGF, ECGF, FGF, or combinations thereof.

23. Platelet rich plasma (PRP) obtained from an allogenic or xenogenic tissue source.

24. The platelet rich plasma of claim 23, wherein the platelet rich plasma is obtained from blood that has been removed from living or cadaveric donors.

25. A method of obtaining platelet rich plasma comprising the steps of:
(a) procuring blood that has been removed from living or cadaveric donors, or both; and
(b) separating platelet rich plasma from other blood components.

26. The method of claim 25, wherein said separating comprises centrifuging said blood.

27. A growth factor composition comprising one or more growth factors that have been extracted from allogenic or xenogenic platelet rich plasma.

28. The growth factor composition of claim 27 comprising PDGF, PAGF, PEGF, TGF-beta, or combinations thereof.

29. The growth factor composition of claim 27, wherein said platelet rich plasma is obtained from blood that has been removed from living or cadaveric donors.

30. An article of manufacture comprising a container and a growth factor composition disposed within said container.

31. The article of manufacture of claim 30, wherein said container is a sealed bottle, vial, or bag.

32. The article of manufacture of claim 30, wherein said growth factor composition comprises EGF, TGF-alpha, TGF-beta, ECGF, GM-CSF, BMP, WGF, VEGF, FGF, IGF, PDGF, PEGF, CDMP or combinations thereof.

33. A method of repairing a wound, defect or other injury comprising contacting an implant with PRP obtained from an allogenic or xenogenic source, or both; and implanting said implant in a patient in need thereof.

34. The method of claim 32, wherein, PRP is obtained from living or cadaveric donors.

35. A method of repairing a wound, defect or other injury comprising contacting an implant with one or more growth factors extracted from PRP obtained from an allogenic or xenogenic source, or both, and implanting said implant in a patient in need thereof.

36. The method of claim 34, wherein PRP is obtained from living or cadaveric donors.

37. A biomedical implant comprised of an osteogenic material and shaped into the form of tape.

38. The biomedical implant of claim 37, wherein said osteogenic material is an osteoinductive substance, an osteoconductive substance, or a combination thereof.

39. The biomedical implant of claim 38, wherein said osteoinductive material is an allograft or xenograft paste (osteogenic or chondrogenic pastes) and demineralized bone matrix (DBM).

40. The biomedical implant of claim 38 wherein said osteoconductive material is hydroxapatite (HA), tricalcium phosphate (TCP), CCC, bioactive glass, bioactive ceramics, or combinations thereof.

41. The biomedical implant of claim 37, wherein said osteogenic material is in association with a carrier.

42. The biomedical implant of claim 41, wherein said carrier is amylopectin, collagen, gelatin, dextran, agarose, or combinations thereof.

43. The biomedical implant of claim 41 wherein said biomedical implant comprises one or more components of the group consisting of carrier associated Growth Factors, carrier associated mineralized particles, morsellized muscle or other tissue, Fibrin powder, Fibrin/plasminogen glue, biomedical plastics, Demineralized Bone Matrix (DBM)/glycerol, cortico cancellous chips (CCC), DBM/pleuronic F127, and DBM/CCC/F127, human tissue/polyesters or polyhydroxy compounds, or polyvinyl compounds or polyamino compounds or polycarbonate compounds or any other suitable viscous carrier.

44. The biomedical implant of claim 37, wherein said biomedical implant is infused with an additive.

45. The biomedical implant of claim 37, wherein said biomedical implant is rolled into a spool.

46. The biomedical implant of claim 37, said biomedical implant comprising two or more layers, wherein said biomedical implant comprises a first layer possessing osteogenic characteristics and a second layer that is inert.

47. The biomedical implant of claim 46, wherein said second layer is comprised of polylactide, poly (alpha-hydroxycarboxylic acids (e.g. poly-D-(−)-3hydroxybutyric acid, poly(lactones), poly(acetals), poly(orthoesters), amylopectin, gelatin, collagen, agarose, dextran, or combinations thereof.

48. The biomedical implant of claim 46, wherein said second layer is derived from tissue.

49. The biomedical implant of claim 48, wherein said tissue is bone, fibrous connective tissue, elastic connective tissue, cartilage, muscle, vasculature, skin, mucous membrane or other soft tissue, or combinations thereof.

50. The biomedical implant of claim 37, wherein said biomedical implant comprises, either exteriorly, interiorly, or both, a support structure.

51. The biomedical implant of claim 50, wherein said support structure is a mesh, suture, wire material, or combinations thereof.

52. The biomedical implant of claim 51, wherein said support structure is made of inert metals such as titanium; inert and/or bioresorbable polymers; demineralized bone, and/or human or nonhuman tissue.

53. A method useful in medical procedures involving fixating bone fractures, ridge augmentation, or sealing a graft implant site comprising the steps of obtaining a biomedical implant comprised of osteogenic material, wherein said biomedical implant is shaped into tape and rolled into a spool; and peeling a portion of said biomedical implant off of said spool.
